(19) United States
(12) Patent Application Publication
(10) Pub. No.: US 2009/0016977 A1
(43) Pub. Date: Jan. 15, 2009

(54) DEODORANT COMPOSITIONS AND METHODS FOR MAKING SAME

(76) Inventors: Barbara Marie Modafari, Mason, OH (US); David Arthur Sturgis, Cincinnati, OH (US); David Frederick Swaile, Cincinnati, OH (US); Songtao Zhou, Mason, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/047,430

(22) Filed: Mar. 13, 2008

Related U.S. Application Data
(60) Provisional application No. 60/918,495, filed on Mar. 16, 2007.

(51) Int. Cl. A61K 8/00

(52) U.S. Cl. 424/65; 424/76.1

(57) ABSTRACT

Deodorant compositions comprising an emulsion including a continuous polar solvent phase and a discontinuous oil phase are provided. The oil phase comprises an oil ingredient and a fragrance material. Methods for making such deodorant compositions are also provided.

### CROSS REFERENCE TO RELATED APPLICATION

This application claims the benefit under 35 USC 119(e) to U.S. Application Ser. No. 60/918,495, filed Mar. 16, 2007.

### FIELD OF THE INVENTION

The present invention is directed to deodorant compositions and methods for making deodorant compositions.

### SUMMARY OF THE INVENTION

Deodorant compositions of the present invention comprise an emulsion including a continuous polar solvent phase and a discontinuous oil phase, and methods for making the same. The oil phase may contain a relatively low to moderate polarity oil ingredient and a fragrance composition comprising high Clog P fragrance components. Without being bound by theory, it is believed that these fragrance components are substantially more soluble in the low to moderately polar high Clog P oil than the continuous polar phase, and thereby “trapped” or held within the emulsion such that the composition may produce a reduced fragrance strength impact when the composition is smelled at shelf or immediately at application to the body. As the polar solvent evaporates or is otherwise removed from the deodorant product film applied to the body, the fragrance material can release from the oil phase to provide an odor masking or fragrance benefit to the consumer.

### DETAILED DESCRIPTION OF THE INVENTION

The present invention may be understood more readily by reference to the following detailed description of illustrative and preferred embodiments. It is to be understood that the scope of the claims is not limited to the specific features, methods, conditions, or parameters described herein, and that the terminology used herein is for the purpose of describing particular embodiments by way of example only and it not intended to be limiting of the claimed invention. Also, as used in the specification including the appended claims, the singular forms “a,” “an,” and “the” include the plural, and reference to a particular numerical value includes at least that particular value, unless the context clearly dictates otherwise. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

All percentages, parts and ratios are based upon the total weight of the compositions of the present invention, unless otherwise specified. All such weights as they pertain to listed ingredients are based on the active level and, therefore, do not include solvents or by-products that may be included in commercially available materials, unless otherwise specified. The term “weight percent” may be denoted as “wt. %” herein.

As used herein, “comprising” means that other steps and other ingredients which do not affect the end result can be added. This term encompasses the terms “consisting of” and “consisting essentially of”. The compositions of the present invention can comprise, consist of, and consist essentially of the elements and limitations of the invention described herein, as well as any of the additional or optional ingredients, components, steps, or limitations described herein.

As used herein, the term “deodorant composition,” includes personal care compositions that comprise fragrance materials that are intended to mask malodor and/or provide a fragrance expression to consumers. The deodorant compositions may also comprise a deodorant active and/or an antiperspirant active. The deodorant compositions may take on a variety of product forms, including, but not limited to, solid sticks, soft solids, creams, body sprays, aerosols, and roll-ons.

The use of Clog P values is well known in the chemical arts as a calculated value that represents the relative affinity that a material has for partitioning between octanol and water, so that a material that partitions more readily into octanol would tend to be more lipophilic and have a higher Clog P value than a material that partitions less readily into octanol. For purposes of defining the deodorant compositions of the present invention or ingredients thereof, Clog P values can be obtained from or calculated by the methods described in Handbook of Physical Properties of Organic Chemicals, Edited by Philip H. Howard and William M. Meylan, CRC Press—Lewis Publishers, 1997.

Clog P values can also be determined by the Pamona Med Chem/Daylight “C LOG P” program, Version 4.42, available from Biobyte Corporation, Claremont, Calif. Other suitable methods for determining Clog P values include the fragment approach described by Hansch and Leo (cf., A. Leo, in Comprehensive Medicinal Chemistry, Vol. 4, C. Hansch, P. G. Sammens, J. B. Taylor and C. A. Ramsden, Eds., p. 295, Pergamon Press, 1990), which description is incorporated herein by reference. Still other suitable methods are described or provided by Daylight Information Systems, Mission Viejo, Calif., Daylight V4.61, Algorithm: V3.05, Database: V16. General information pertaining to Clog P values and methodologies are described in Chemical Reviews, 93(4), 1993, 1281-1306. As used herein, Clog P values include calculated and measured log P values.

As used herein, the term “high Clog P” includes Clog P values that are greater than 3, greater than 4, greater than 5, and greater than 6.

Exemplary methods of manufacturing deodorant compositions will now be described, including a description of the steps employed in the methods and illustrative materials that can be utilized in the methods to make deodorant compositions according to the present invention.

### I. Exemplary Method One

Exemplary method one comprises the steps of: (a) forming a first process stream comprising a liquid carrier material and having a first temperature; (b) forming a second process stream comprising water, a water insoluble oil, a surfactant, and a fragrance material, the second process stream having a second temperature that is different from the first temperature by more than about 2° C.; and (c) combining the first process stream and the second process stream to form a deodorant composition and depositing the composition into a container. Deodorant compositions manufactured by this method comprise an emulsion including the water insoluble oil and fragrance material disposed within a portion of the water.

In some embodiments, the first temperature is higher than the second temperature. In other embodiments, the opposite scenario exists. The temperature difference between the first and second streams can be more than 2° C., 10° C., 20° C., 30° C., 40° C., or 60° C.

A. First Process Stream

The first process stream includes a liquid carrier material. A representative, non-limiting, list of suitable liquid carrier materials includes water, ethanol, propylene glycol, dipropylene glycol, tripropylene glycol, glycerin, methyl propylene glycol, butylene glycol, polyethylene glycol, polypropylene glycol, and mixtures thereof. The first process stream may optionally include a thickening agent. The thickening agent can help provide the final composition with the desired viscosity, rheology, texture and/or product hardness, or to otherwise help suspend any dispersed solids or liquids within the composition. The term “thickening agent” may include any material known or otherwise effective in providing suspending, gelling, viscosifying, solidifying or thickening properties to the composition or which otherwise provide structure to the final product form. These thickening agents may include gelling agents, polymeric or nonpolymeric agents, inorganic thickening agents, or viscosifying agents. The thickening agents may include organic solids, silicone solids, crystalline or other gellants, inorganic particulates such as clays or silicas, or combinations thereof.

The concentration and type of the thickening agent selected for use in compositions of the present invention will vary depending upon the desired product form, viscosity, and hardness. The thickening agents suitable for use herein, may have a concentration range from at least about 0.1%, at least about 3%, or at least about 5%, but no more than about 35%, no more than about 20%, or no more than about 10%, by weight of the composition.

Non-limiting examples of suitable gelling agents include fatty acid gellants, salts of fatty acids, hydroxyl acids, hydroxyl acid gellants, esters and amides of fatty acid or hydroxyl fatty acid gellants, cholesterolic materials, dibenzylidene alditols, lanolinolic materials, fatty alcohols, triglycerides, sucrose esters such as SEFA behenate, inorganic materials such as clays or silicas, other amide or polyamide gellants, and mixtures thereof.

B. Second Process Stream

The second process stream includes water, a water insoluble oil, and a surfactant. The second process stream also may optionally include a thickening agent. Exemplary water insoluble oils include mineral oil, petrolatum, isoparraffins, PPG-14 butyl ether, isopropyl myristate, butyl stearate, cetyl ocatnoate, butyl myristate, C12-C15 alkyl benzoate (e.g., Finsolv-TN™ from Finetex), octyldodecnaol, isostearyl isostearate, octododecyl benzoate, isostearyl lactate, isostearyl palmitate, and isobutyl stearate. A representative, non-limiting list of suitable surfactants includes steareth-2, steareth-21, stearyl alcohol, C20-C40 alcohol, C20-C40 pareth 40, and mixtures thereof. Other water insoluble oils and surfactants may also be used in methods of the present invention.

The second process stream also includes a fragrance material, which can comprise one or more fragrance components. Although a variety of different fragrance components can be employed, it is preferred to have a fragrance material that includes at least some high Clog P fragrance components. Table 1 below provides an exemplary list of such fragrance components.

TABLE 1
Exemplary Fragrance Component List

| | Clog P |
|---|---|
| Fragrance Component | value |
| Allyl cyclohexane propionate | 3.935 |
| Ambrettolide | 6.261 |
| Amyl benzoate | 3.417 |
| Amyl cinnamate | 3.771 |
| Amyl cinnamic aldehyde | 4.324 |
| Amyl cinnamic aldehyde dimethyl acetal | 4.033 |
| Iso-amyl salicylate | 4.601 |
| Aurantiol (Trade name for Hydroxycitronellal- | 4.216 |
| methylanthranilate) | |
| Benzyl salicylate | 4.383 |
| para-tert-Butyl cyclohexyl acetate | 4.019 |
| Iso butyl quinoline | 4.193 |
| beta-Caryophyllene | 6.333 |
| Cadinene | 7.346 |
| Cedrol | 4.530 |
| Cedryl acetate | 5.436 |
| Cedryl formate | 5.070 |
| Cinnamyl cinnamate | 5.480 |
| Cyclohexyl salicylate | 5.265 |
| Cyclamen aldehyde | 3.680 |
| Diphenyl methane | 4.059 |
| Diphenyl oxide | 4.240 |
| Dodecalactone | 4.359 |
| Iso E Super (Trade name for 1-(1,2,3,4,5,6,7,8- | 3.455 |
| Octahydro-2,3,8,8-tetramethyl-2-naphthalenyl)- | |
| ethanone) | |
| Ethylene brassylate | 4.554 |
| Ethyl undecylenate | 4.888 |
| Exaltolide (Trade name for 15-Hydroxyentadecanloic | 5.346 |
| acid, lactone) | |
| Galaxolide (Trade name for 1,3,4,6,7,8-Hexahydro- | 5.482 |
| 4,6,6,7,8,8-hexamethylcyclopenta-gamma-2-benzopyran) | |
| Geranyl anthranilate | 4.216 |
| Geranyl phenyl acetate | 5.233 |
| Hexadecanolide | 6.805 |
| Hexenyl salicylate | 4.716 |
| Hexyl cinnamic aldehyde | 5.473 |
| Hexyl salicylate | 5.260 |
| Alpha-Irone | 3.820 |
| Lilial (Trade name for para-tertiary-Butyl-alpha- | 3.858 |
| methyl hydrocinnamic aldehyde) | |
| Linalyl benzoate | 5.233 |
| Methyl dihydrojasmone | 4.843 |
| Gamma-n-Methyl ionone | 4.309 |
| Musk indanone | 5.458 |
| Musk tibetine | 3.831 |
| Oxahexadecanolide-10 | 4.336 |
| Oxahexadecanolide-11 | 4.336 |
| Patchouli alcohol | 4.530 |
| Phantolide (Trade name for 5-Acetyl-1,1,2,3,3,6- | 5.977 |
| hexamethyl indan) | |
| Phenyl ethyl benzoate | 4.058 |
| Phenylethylphenylacetate | 3.767 |
| Phenyl heptanol | 3.478 |
| Alpha-Santalol | 3.800 |
| Thibetolide (Trade name for 15-Hydroxypentadecanoic | 6.246 |
| acid, lactone) | |
| Delta-Undecalactone | 3.830 |
| Gamma-Undecalactone | 4.140 |
| Vetiveryl acetate | 4.882 |
| Ylangene | 6.268 |

In some embodiments, at least 25% or 50%, by weight, of the fragrance material comprises fragrance components that have a Clog P value that is greater than about 3. For these embodiments, the water insoluble oil may also have a Clog P value that is greater than about 3.

As noted above, the emulsion may be formed prior to combining the first and second process streams. Thus, in this scenario, the second stream mixture itself is processed with an appropriate apparatus, such as a mill, to form an emulsion.

The first and second streams are combined with one another and mixed to form the deodorant composition. Various known mixing devices can be employed, such as, for example, a static mixer. The deodorant composition is deposited into a suitable container (which may be a temporary container or the container in which the composition is sold), and can then either be allowed to cool or cooled with a cooling device down to room temperature or another targeted temperature.

### II. Exemplary Method Two

Exemplary method two comprises the steps of: (a) forming an emulsion comprising a polar solvent phase, an oil phase that includes a fragrance component and that is disposed within the polar solvent phase, and a surfactant; (b) forming a pre-mix comprising a liquid carrier and a gellant; (c) heating the pre-mix to a first temperature that is capable of melting the gellant; (d) cooling the premix to a second temperature that is just above the onset of crystallization of the gellant but is below the melting point of the emulsion; (e) adding the emulsion to the pre-mix at or around the second temperature to form the deodorant composition; and (f) depositing the deodorant composition into a container.

A. Emulsion

An emulsion is formed comprising a continuous polar solvent phase and a discontinuous oil phase. A representative, non-limiting, list of suitable polar solvents includes water, ethanol, propylene glycol, dipropylene glycol, tripropylene glycol, glycerin, methyl propylene glycol, butylene glycol, polyethylene glycol, polypropylene glycol, and mixtures thereof.

The oil phase may contain a variety of different oil ingredients, including, for example, organic liquid carriers such as mineral oil, petrolatum, isohexadecane, isododecane, various other hydrocarbon oils, and mixtures thereof. Specific non-limiting examples of suitable branched chain hydrocarbon oils include isoparaffins available from Exxon Chemical Company as Isopar C™ (C7-C8 Isoparaffin), Isopar E™ (C8-C9 Isoparaffin), Isopar G™ (C10-11 Isoparaffin), Isopar H™ (C11-C12 Isoparaffin), Isopar L™ (C11-C13 Isoparaffin), Isopar M™ (C13-C14 Isoparaffin), and combinations thereof. Other non-limiting examples of suitable branched chain hydrocarbons include Permethyl™ 99A (isododecane), Permethyl™ 102A (isoeicosane), Permethyl™ 101A (isohexadecane), and combinations thereof. The Permethyl™ series are available from Preperse, Inc., South Plainfield, N.J., U.S.A. Other non-limiting examples of suitable branched chain hydrocarbons include petroleum distillates such as those available from Phillips Chemical as Soltrol™ 130, Soltrol™ 170, and those available from Shell as Shell Sol™ 70, -71, and -2033, and mixtures thereof.

Examples of other suitable oil ingredients include the Norpar™ series of paraffins available from Exxon Chemical Company as Norpar™ 12, -13, and -15; octyldodecanol; butyl stearate; diisopropyl adipate; dodecane; octane; decane; C1-C15 alkanes/cycloalkanes available from Exxon as Exxsol™ D80; C12-C15 alkyl benzoates available as Finsolv-TN™ from Finetex; and mixtures thereof. Other suitable liquid carriers include benzoate co-solvents, cinnamate esters, secondary alcohols, benzyl acetate, phenyl alkane, and combinations thereof.

One preferred list of oil ingredients includes the following hydrocarbon, high Clog P liquids: mineral oil, petrolatum, isoparraffins, PPG-14 butyl ether, isopropyl myristate, butyl stearate, cetyl ocatnoate, butyl myristate, C12-C15 alkyl benzoate (e.g., Finsolv-TN™ from Finetex), octyldodecnaol, isostearyl isostearate, octododecyl benzoate, isostearyl lactate, isostearyl palmitate, and isobutyl stearate.

The oil phase contains one or more fragrance components. Table 1, above, lists a number of suitable fragrance components that may be employed in the oil phase. Other fragrance components may also be used, with a preference towards those having a Clog P value that is greater than about 3.

A representative, non-limiting list of emulsifying surfactants includes steareth-2, steareth-21, stearyl alcohol, C20-C40 alcohol, C20-C40 pareth 40, and mixtures thereof.

B. Pre-Mix

A pre-mix is formed comprising a liquid carrier and a gellant. Numerous different liquid carrier materials may be employed, including, for example, water, ethanol, propylene glycol, dipropylene glycol, tripropylene glycol, glycerin, methyl propylene glycol, butylene glycol, polyethylene glycol, polypropylene glycol, and mixtures thereof. Non-limiting examples of suitable gelling agents include fatty acid gellants, salts of fatty acids, hydroxyl acids, hydroxyl acid gellants, esters and amides of fatty acid or hydroxyl fatty acid gellants, cholesterolic materials, dibenzylidene alditols, lanolinolic materials, fatty alcohols, triglycerides, sucrose esters such as SEFA behenate, inorganic materials such as clays or silicas, other amide or polyamide gellants, and mixtures thereof.

The pre-mix is heated to a first temperature that is capable of melting the chosen gellant. For many known gellants, a temperature equal to or greater than about 85° C. is sufficient to effectively melt the gellant. The pre-mix is then cooled to a second temperature that is just above the onset of crystallization of the gellant, but below the melting point of the emulsion. By way of example only, the second temperature may be in the range of 60° C. to 80° C. The pre-mix and emulsion are combined at or around this second temperature to form a deodorant composition, which can then be deposited into a container.

Exemplary method two may be used to make the following deodorant compositions within the scope of the present invention: 1) a deodorant composition, comprising a gellant and an emulsion, the emulsion comprises a polar solvent phase and an oil phase disposed therein, and the oil phase comprises a water insoluble oil, wherein the emulsion does not phase separate when the composition is heated above the onset of crystallization of the gellant; and 2) a deodorant composition, comprising a gellant and an emulsion, the emulsion comprises a polar solvent phase and an oil phase disposed therein, and the oil phase comprises a water insoluble oil, wherein the emulsion does not phase separate when the composition is heated to 60° C., 65° C., or 70° C.

### EXAMPLES

The following examples further describe and demonstrate embodiments within the scope of the present invention. The examples are given solely for the purpose of illustration and are not to be construed as limitations of the present invention as many variations thereof are possible without departing from the spirit and scope of the invention.

Deodorant Composition Examples A-D. The numerical values in the table are in percent by weight of the total composition.

| Ingredient | A | B | C | D |
|---|---|---|---|---|
| dipropylene glycol | 45 | 30 | 20 | 45 |
| propylene glycol | 22 | | 22 | 22 |
| tripopylene glycol | | | 25 | |
| glycerine | | 10 | | |
| PEG -8 | | 20 | | |
| water | QS | QS | QS | QS |
| 50% aqueous aluminum | | | | |
| chlorohydrate | | | | |
| sodium stearate | 5.5 | 5.5 | 5.5 | 5.5 |
| tetra sodium EDTA | 0.05 | 0.05 | 0.05 | 0.05 |
| steareth-2 | 2.2 | | | |
| steareth-21 | 1.0 | 1.5 | | |
| stearyl alcohol | | 2.0 | | 2.0 |
| C20-C40 alcohol ^{1.} | | | 2.0 | |
| C20-C40 pareth 40 ^{2.} | | | 1.0 | 1.0 |
| petrolatum | 1.75 | | 1.75 | 1.75 |
| mineral oil | | 1.75 | | |
| dye | 0.002 | 0.002 | 0.002 | 0.002 |
| high C logP fragrance | 0.2-2 | 0.2-2 | 0.2-2 | 0.2-2 |
| composition ^{3.} | | | | |
| base fragrance | 0.5-2 | 0.5-2 | 0.5-2 | 0.5-2 |

QS - indicates that this material is used to bring the total to 100%.
^{1.} Available from New Phase Technologies as Performachol 350.
^{2.} Available from New Phase Technologies as Performathox 480.
^{3.} This fragrance comprises at least 50% fragrance components with C log P values greater than 3

Examples A and B can be made by a dual stream process as follows:
Hot Stream Create an oil phase by combining steareth-2 or stearyl alcohol, steareth-21, petrolatum, and the high Clog P fragrance composition in a container and heat to about 65° C. Create an aqueous phase by combining the water, sodium stearate, EDTA, and about 10% of the glycols in a separate container and heating to about 85° C. Cool the aqueous solution to about 75° C. Once the oil phase has melted and is homogenous, add it to the aqueous phase with agitation.
Cold Stream Combine the remaining polar emollients, base fragrance, and dye in a separate container and mix until homogenous.
Combine the hot stream and the cold stream via a two-stream filler, at approximately a 1:1 ratio and mix (e.g., with a static mixer) to form the deodorant composition. Charge the deodorant composition into a suitable container and cool.
Examples C and D can be made as follows: Create an oil phase by combining the C20-40 alcohol or stearyl alcohol, C20-C40 pareth 40, petrolatum, and the high Clog P fragrance composition in a container and heat to about 85° C. In a separate container, heat about half of the water to about 85° C. Once the oil phase is melted and homogenous, slowly add it to the heated water and agitate the mixture to form an emulsion. Allow the emulsion to cool slowly to ambient temperature while stirring. Allow the emulsion to equilibrate for at least 6 hours, and preferably, for at least 24 hours. Combine the glycols, the remaining water, sodium stearate, EDTA, and dye in a suitable container and heat to about 85° C. Once the solids are dissolved, add the base fragrance and the emulsion to the solution with agitation. Cool the solution to about 70° C. and pour into an appropriate container.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of manufacturing a deodorant composition, comprising the steps of:
(a) forming a first process stream comprising a liquid carrier material and having a first temperature;
(b) forming a second process stream comprising water, a water insoluble oil, a surfactant, and a fragrance material, the second process stream having a second temperature that is different from the first temperature by more than about 2° C.; and
(c) combining the first process stream and the second process stream to form a deodorant composition and depositing the deodorant composition into a container,
wherein the deodorant composition comprises an emulsion including the water insoluble oil and fragrance material disposed within a portion of the water.

2. The method of claim 1, wherein the first temperature is higher than the second temperature.

3. The method of claim 1, wherein the second temperature is higher than the first temperature.

4. The method of claim 1, wherein the second temperature is different from the first temperature by more than about 10° C.

5. The method of claim 1, wherein the second temperature is different from the first temperature by more than about 20° C.

6. The method of claim 1, wherein the second temperature is different from the first temperature by more than about 30° C.

7. The method of claim 1, wherein the second temperature is different from the first temperature by more than about 40° C.

8. The method of claim 1, wherein either the first temperature or the second temperature is less than about 60° C.

9. The method of claim 1, wherein the water insoluble oil comprises petrolatum.

10. The method of claim 1, wherein the water insoluble oil comprises mineral oil.

11. The method of claim 1, wherein either the first process stream or the second process stream contains a gellant.

12. The method of claim 11, wherein the gellant comprises a fatty acid salt.

13. The method of claim 1, wherein the liquid carrier material comprises a glycol material.

14. The method of claim 1, wherein each of the fragrance material and the water insoluble oil has a Clog P value that is greater than about 3.

15. The method of claim 1, wherein at least 25%, by weight, of the fragrance material comprises fragrance components that have a Clog P value that is greater than about 3, and the water insoluble oil has a Clog P value that is greater than about 3.

16. The method of claim 1, wherein at least 50%, by weight, of the fragrance material comprises fragrance components that have a Clog P value that is greater than about 3, and the water insoluble oil has a Clog P value that is greater than about 3.

17. A method of manufacturing a stick deodorant composition, comprising the steps of:
(a) forming an emulsion comprising a polar solvent phase, an oil phase that includes a fragrance component and that is disposed within the polar solvent phase, and a surfactant;
(b) forming a pre-mix comprising a liquid carrier and a gellant;
(c) heating the pre-mix to a first temperature that is capable of melting the gellant;
(d) cooling the premix to a second temperature that is just above the onset of crystallization of the gellant but is below the melting point of the emulsion;
(e) adding the emulsion to the pre-mix at or around the second temperature to form the deodorant composition; and
(f) depositing the deodorant composition into a container.

18. The method of claim 17, wherein the first temperature is greater than or equal to about 85° C.

19. The method of claim 17, wherein the second temperature is from about 60° C. to about 80° C.

20. A deodorant composition, comprising a gellant and an emulsion, the emulsion comprises a polar solvent phase and an oil phase disposed therein, and the oil phase comprises a water insoluble oil, wherein the emulsion does not phase separate when the composition is heated above the onset of crystallization of the gellant.

21. A deodorant composition, comprising a gellant and an emulsion, the emulsion comprises a polar solvent phase and an oil phase disposed therein, and the oil phase comprises a water insoluble oil, wherein the emulsion does not phase separate when the composition is heated to 60° C.

22. The composition of claim 21, wherein the emulsion does not phase separate when the composition is heated to 65° C.

23. The composition of claim 21, wherein the emulsion does not phase separate when the composition is heated to 70° C.
